(19) United States
(12) Patent Application Publication
(10) Pub. No.: US 2020/0248321 A1
(43) Pub. Date: Aug. 6, 2020

(54) ELECTROCHEMICAL DEHYDROGENATION OF ALKANES TO ALKENES

(71) Applicant: ExxonMobil Research and Engineering Company, Annandale, NJ (US)

(72) Inventors: Robert B. Larson, Houston, TX (US); Benjamin A. McCool, Annandale, NJ (US)

(21) Appl. No.: 16/742,068

(22) Filed: Jan. 14, 2020

Related U.S. Application Data
(60) Provisional application No. 62/801,946, filed on Feb. 6, 2019.

(51) Int. Cl. C25B 3/04; C25B 11/03; C25B 13/04; C07C 5/32; C25B 9/08; C07C 11/04; C07C 11/06

(52) U.S. Cl. CPC C25B 3/04; C25B 11/035; C25B 13/04; C07C 11/06; C25B 9/08; C07C 11/04; C07C 5/32

(57) ABSTRACT

A process and apparatus useful for continuously contacting an alkane feed at a temperature within a range from 300° C. to 600° C. and a pressure within the range from 50 psig to 500 psig with one or more reactive ceramic membrane to form an alkene with some remaining alkane and hydrogen, the hydrogen in physical isolation from the alkane and alkene, separating the alkene from the remaining alkane, and recycling the alkane to contact the reactive ceramic membrane.

### CROSS-REFERENCE TO RELATED APPLICATIONS

This application claims the benefit of U.S. Provisional Application No. 62/801,946, filed Feb. 6, 2019, the entire contents of which are incorporated herein by reference.

### FIELD OF THE INVENTION

The present invention is directed to the electrochemical dehydrogenation of alkanes to alkenes, and in particular to the production of ethylene from ethane using solid membrane electrochemical reactors.

### BACKGROUND

Ethane cracking is an energy intensive process, in part because of the extreme temperature and pressure differences that exist between the reaction and separation sections. It is possible to achieve a significant reduction in energy consumption using a novel reactor technology that operates at low temperature and elevated pressure.

Electrochemical ethane dehydrogenation using solid proton conductive materials is known. Ethane dehydrogenation takes place on the membrane surface, driven a combination of thermal and electrical energy. Protons are removed from an ethane molecule, transported across the membrane, where they recombine at the anode surface forming a high purity hydrogen product. On the cathode side of the membrane ethane is converted to ethylene at low temperature (about 400° C.), with 100% selectivity. In addition it is possible to run this system at high pressure, as hydrogen separation counters the negative impact of pressure on dehydrogenation rates. This membrane, H₂ removal, and high selectivity provide a pathway to significantly improve the economics of ethylene production. The present disclosure is directed to such a proposed flow scheme, which takes advantage of this reactor technology in a way that significantly improve process economics.

References of interest include: D. Ding et al., “A novel low-thermal-budget approach for the co-production of ethylene and hydrogen via the electrochemical non-oxidative deprotonation of ethane,” in 11 ENERGY ENVIRON. SCI. 1710 (2018); and H. Iwahara et al., “High temperature-type proton conductive solid oxide fuel cells using various fuels,” in 16 J. APPL. ELECTROCHEMISTRY 663 (1986).

### BRIEF DESCRIPTION OF DRAWING

FIG. 1 is a simplified engineering diagram of an embodiment of the apparatus described herein, and the process flow of the process described herein.

FIG. 2 is a simplified engineering diagram of another embodiment of the apparatus described herein, and the process flow of the process described herein.

### SUMMARY

Disclosed is process comprising (or consisting of, or consisting essentially of) continuously contacting an alkane feed at a temperature within a range from 300, or 350, or 400° C. to 450, or 500, or 600° C. and a pressure within the range from 50, or 100, or 200, or 250, or 300 psig to 400, or 450, or 500 psig with one or more reactive ceramic membranes to form an alkene with some remaining alkane and hydrogen, the hydrogen in physical isolation from the alkane and alkene, separating the alkene from the remaining alkane, and recycling the alkane to contact the reactive ceramic membrane.

Also disclosed is an apparatus comprising (or consisting of, or consisting essentially of) a membrane reactor comprising one or more reactive ceramic membranes, the membrane reactor having to an inlet for the introduction of heated alkane feed, an outlet for the removal of hydrogen, and an outlet for the recirculation of unreacted alkane and alkene product first to a cooler then to at least one separating column to allow recirculation of alkane back to the membrane reactor.

### DETAILED DESCRIPTION

Disclosed is an improved apparatus and process for conversion of alkanes to alkenes via electrochemically driven dehydrogenation. The proposed dehydrogenation apparatus operation would begin in any embodiment with the fractionation of lower alkanes such as methane from the target alkane, such as ethane, feedstock. This would typically be performed downstream of a pyrolysis furnace, however, by eliminating methane formation in the reactor, it is desirable to perform this separation on the smaller feed stream. The demethanizer column bottoms is then combined with recycle ethane, which is then heated to 400° C. using both a combined feed exchanger and fired heater. The reactor, preferably a membrane reactor, comprises a proton conducting electrochemical cell in which electrical energy is used to promote low temperature dehydrogenation. Under these conditions, the reaction selectivity approaches 100%, reducing both feed loss and the requirements for downstream cleanup. The membrane reactor separates hydrogen, giving both a high purity hydrogen product stream, as well as a hydrocarbon stream that is free of hydrogen. The effluent from the reactor is cooled in a combined feed exchanger, preferably cooled such as in a cooler, and separated in a conventional deethanizer and ethane (C2) splitter configuration to recover the target alkane such as ethylene.

As used herein, “Group” or “Groups” refers to vertical groups of elements in the Periodic Table of Elements as in HAWLEY'S CONDENSED CHEMICAL DICTIONARY (John Wiley, 1997). “Rare Earth” refers to elements on the Periodic Table also referred to as the ‘lanthanide series.’

As used herein, unless stated otherwise, a “column” or “separating column” refers to a distillation or ‘rectification’ apparatus that effects the separation of two or more components in a mixture with different chemical attributes such as polarity, condensation temperatures and/or boiling point temperatures from one another and may take on any configuration as is known in the art. Such columns typically have at least a “bottoms” and “overhead” which refers to heavier/higher boiling point components as opposed to lighter/lower boiling point components, respectively, but may also refer to other chemical attributes that cause one components of the mixture to separate from the other component(s), such as differences in polarity or molecular diffusion.

Thus, in any embodiment is process comprising (or consisting of, or consisting essentially of) continuously contacting an alkane feed at a temperature within a range from 300, or 350, or 400° C. to 450, or 500, or 600° C. and a pressure within the range from 50, or 100, or 200), or 250, or 300 psig to 400, or 450, or 500 psig with a reactive ceramic membrane to form an alkene with some remaining alkane and hydrogen, the hydrogen in physical isolation from the alkane and alkene, separating the alkene from the remaining alkane, and recycling the alkane to contact the reactive ceramic membrane.

By “physical isolation”, what is meant is that the hydrogen is separated from the alkanes and alkene products by a physical barrier such as the reactive ceramic membrane itself such that the alkanes and alkenes cannot pass through to the side of the membrane where the hydrogen resides, and the hydrogen can be continuously removed to prevent or diminish its diffusion through the membrane.

By “continuous contacting”, what is meant is that fresh and/or recycled reactant material such as alkane is provided at a non-zero rate within a reactor and withdrawn from that reactor, such as by a steady flow a liquid material passing across a solid interface from one or more entrance points to one or more exit points, such contacting preferably allowing the reactant to reactively engage with such solid interface, such as the reactive ceramic membrane, to convert alkanes to alkenes and hydrogen.

In any embodiment the process is carried out in an apparatus comprising (or consisting of, or consisting essentially of) a membrane reactor comprising at least one a reactive ceramic membrane, the membrane reactor having an inlet for the introduction of heated alkane feed, a first outlet for the removal of hydrogen, and an second outlet for the recirculation of unreacted alkane and alkene product first to a cooler then to at least one separating column to allow recirculation of alkene back to the membrane reactor.

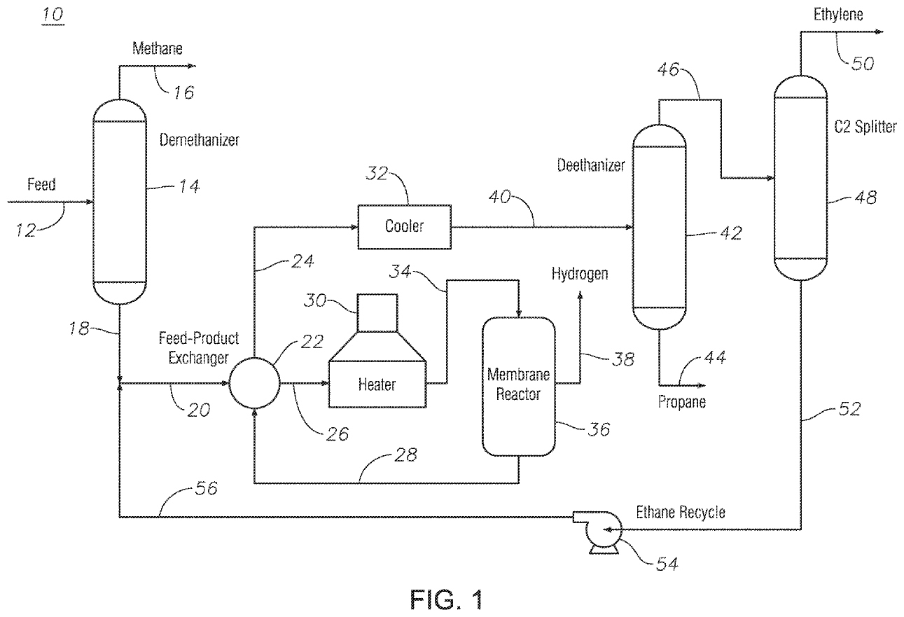

An example of a dehydrogenation apparatus useful in the present process is shown in the FIG. 1, where a hydrocarbon feed 12 is feed into dehydrogenation apparatus 10, such feed comprising a desirable alkane such as ethane that can be dehydrogenated to the target alkene such as ethylene. In any embodiment, a hydrocarbon feed 12 is provided and/or maintained at a pressure within a range from 400, or 500, or 600 psig to 1200, or 1400, or 1800 psig, and a temperature within a range from 0, or 10, or 20° C. to 40, or 50, or 80° C. The feed 12 may be transferred to a demethanizer 14, a separation unit such as a distillation column, resulting in a light alkane stream 16 such as methane and a heavy alkane stream 18 such as ethane, propane, and minor amounts of higher alkanes. The stream 18 may then be transferred via feed 20 to a feed-product exchanger 22 where a portion of the feed of the feed goes via feed 26 to a heater 30, heating that portion of the feed before it is transferred via feed 34 to a membrane reactor 36, and some of the feed 24 is transferred to a cooler 32, such portion of feed 40 then transferred to a deethanizer 42 that can separate heavier, non-targeted alkanes such as propane 44, and lighter alkanes such as ethane through feed 46.

Alternately, in any embodiment, the membrane reactor 36 itself may be heated to influence the temperature of the feed and product therein. Such heating may be achieved by locating the membrane reactor inside of a heating apparatus, or having a heating coil or other exchanger lining the walls or exterior of the membrane reactor.

In the membrane reactor 36, the heated feed contacts the one or more reactive ceramic membranes while an electrical current is applied across the one or more reactive ceramic membranes to convert, for example, ethane to ethylene, and hydrogen is then removed via feed 38 while the ethylene and unreacted ethane (reactor effluent) is removed via feed 28 back to the feed-product exchanger 22. The heater 26 preferably heats the feed 34 to a temperature within a range from 300, or 350, or 400° C. to 450, or 500, or 600° C., the feed preferably maintained at a pressure within a range from 50, or 100, or 200, or 250, or 300 psig to 400, or 450, or 500 psig.

Referring still to the FIG. 1, as for the portion of the feed 22 that is transferred to the cooler 32, that feed is preferably cooled to a temperature within a range from −30, or −20, or −10, or 0° C. to 10, or 20, or 30, or 40° C. In any case, recycled alkane/alkene reactor effluent feed 28 that is recycled from the membrane reactor 36 will be transferred to the feed-product exchanger 22 that will then transfer the mixture to the cooler, then the deethanizer 42, then to a C2 splitter 48 via feed 46 to separate the desired alkene such as ethylene 50, and recycle the alkanes via feed 52, desirably driven by a pump or other pressure generating device 54 back via feed 56 to the feed-product exchanger 22.

In any embodiment, the hydrocarbon feed 42 is at a pressure within a range from 50, or 100, or 200, or 250, or 300 psig to 350, or 400 psig and a temperature within a range from −30, or −20, or −10, or 0° C. to 10, or 20, or 30, or 40° C. In any embodiment, the hydrocarbon feed 48 is at a pressure within a range from 150, or 200 psig to 300, or 350 psig and a temperature within a range from −40, or −30, or −20° C. to 10, or 20, or 30, or 40° C.

The feed 12 can be any desirable hydrocarbon feed from any convenient source, whether it is an adjoining refining or conversion plant, or shipped in materials from a distant source. In a preferred embodiment, the feed 12 comprises at least 80, or 85, or 90, or 95 wt/o, by weight of the feed, or ethane, the remaining portion comprising methane, propylene, and butane.

In any embodiment, the hydrogen 38 is a high purity hydrogen that is at least 99% pure and needs no further refinement for further use in chemical processes such as syngas processes, hydrogenation processes, and other desirable chemical conversions.

The membrane reactor is the component of apparatus 10 that carries out the electrochemically driven dehydrogenation of alkanes such as ethane to the corresponding alkene, which for example consumes ethane to generate ethylene and hydrogen. Hydrogen is separated within the reactor and recovered as a high purity product. Unconverted ethane and product ethylene do not cross the membrane, and the thermal energy from this stream is recovered in the feed-product exchanger. An example of a desirable membrane reactor would be those made by Praxair™ wherein one or more tubes lined with reactive ceramic membrane, or layered throughout the tube(s) therein, would allow heated feed 34 to come into contact with the ceramic membrane(s), wherein an alkane would come into contact with the reactive ceramic membrane and react therewith, allowing alkenes to continue to flow through the tube(s) and out of the system in reactor effluent feed 28, while hydrogen generated from the reaction of the alkane with the membrane would pass through the membrane and be physically separated, such as by having the tubes within a closed chamber that captures the hydrogen and allows its collection to be carried away in feed 38.

The reactive ceramic membrane is preferably an electrochemical membrane based on or comprising those described by D. Ding et al., in 11 ENERGY ENVIRON. SCI. 1710 (2018); or H. Iwahara et al., in 16 J. APPL. ELECTROCHEMISTRY 663 (1986), referenced above. Such membranes may have at least a ceramic anode, a ceramic cathode, and/or a ceramic electrolytic separator between the anode and cathode. Desirably, the alkane such as ethane will contact one face of the membrane, react by extraction of two protons, and form electrochemically hydrogen physically separated from the dehydrogenation product, such as ethylene.

In any embodiment, the reactive ceramic membrane comprises (or consists essentially of) one or more proton-conducting electrolyte films, one or more porous anode supports and one or more porous cathodes. The alkane can be continuously fed to the anode(s) and electrochemically deprotonated into the corresponding alkene and protons when an electrical field is applied across the layers. The generated protons then transfer through a dense proton-conducting membrane (electrolyte layer) to the cathode(s) where they combine with electrons and form high-purity hydrogen in physical isolation from the alkanes and alkenes formed therefrom. Preferably, the reactive ceramic membrane is impermeable to the alkanes and alkenes generated therefrom, not allowing these molecules to pass or diffuse through the thickness of the membrane from one physical side to the other, allowing the hydrogen generated from the electrochemical reaction to be in isolation from the alkanes and alkenes, and hence removed separately from the membrane reactor.

In any embodiment, the reactive ceramic membranes comprise Group 2-Rare Earth complex oxides, examples of which include Group 2 complexes with cerium, yttrium, and/or cerium and/or ytterbium oxides, and which preferably include metals and/or metal oxides of Group 4 to 10 metals such as nickel, iron and/or zirconium, depending on whether the membrane is serving as a anode, cathode, or electrolytic separator. In any embodiment, the reactive ceramic membrane comprises at least one cathode layer, and at least one anode layer, and an electrolyte barrier between the at least one cathode and at least one anode. Each anode and/or cathode layer may be the same or different with respect to its chemical identity, thickness, or both. Also, in any embodiment, an inert metal layer such as platinum may serve as an electrode material. Preferably, the anode(s), cathode(s) and electrolyte layers are porous to allow hydrogen atoms and hydrogen molecules to flow through. In any embodiment, a layer of anode/electrolyte/cathode may be within a range from 5, or 10, or 20 μm to 50, or 100, or 200, or 1000 μm in average thickness.

In any embodiment, the role of the demethanizer column (or “demethanizer”) is to separate the lightest (lower molecular weight) portions of an alkane feed such as methane, which is found in most fresh feeds, from the ethane, which is converted to produce ethylene. The demethanizer may be a small, high pressure column, which makes use of the high pipeline pressures, to reduce the utility consumption. The objective is to remove methane to prevent it from contaminating the ethylene product. Typically this separation would be performed downstream of the reactor, to capture any methane formed in the process. However, it is advantageous to perform this separation upstream of the reactor, which can only be done if the reaction does not produce methane.

In any embodiment, a demethanizer column is additionally, or alternatively located just downstream of the cooler 32 such that the cooled product 40 first enters a demethanizer, followed by flowing into a deethanizer column. This would remove any methane that might be generated in the membrane reactor in addition to any methane that was in the feed 12.

In any embodiment, there is a compressor and/or flash drum between the cooler 32 and the deethanizer column 42, or between the cooler and the demethanizer column (if present).

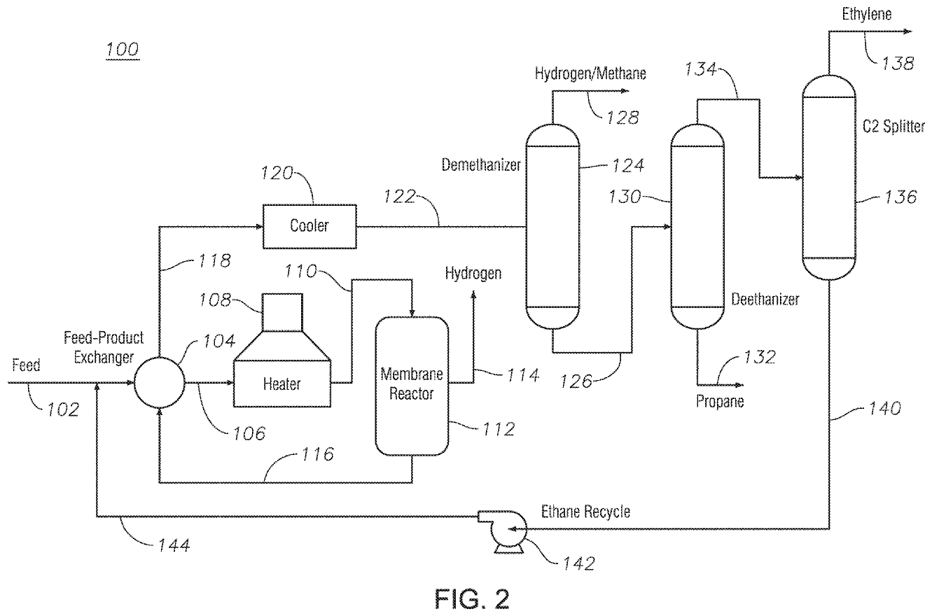

Another example of a dehydrogenation apparatus useful in the present process is shown in FIG. 2 wherein the demethanizer is located downstream of the membrane reactor and/or cooler. A feed 102 (similar to feed 12 with respect to FIG. 1) is fed into apparatus 100 directly to the feed-product exchanger 104 as the first significant process step, wherein the feed 106 is then heated in heater 108 and is fed through feed 110 to the membrane reactor 112. Hydrogen may be removed as feed 114, and unreacted alkane and alkene product fed through feed 116 back to the feed-product exchanger 104 to be fed through feed 118 to a cooler 120, or to otherwise be cooled in some manner, such cooled feed 122 then flowing into a demethanizer 124, separating out any remaining hydrogen and methane 128, the heavies then carried in feed 126 to a deethanizer 130 to separate out propane (as well as propylene and other heavier hydrocarbons (C3+) that may result, for example, from oligomerization of alkenes) 132 and feed the lighter alkanes and alkenes through feed 134 to the C2 splitter 136 where the light alkenes such as ethylene 138 is removed. The heavy alkanes such as ethane is recycled in feed 140 to a pump or compressor 142 to feed 144 and back to either the feed 102 or directly into the feed-product exchanger 104. This process is similar to that shown in FIG. 1 except for the arrangement of the step and apparatus component related to the demathanizer 124.

The feed-product heat exchanger contacts cold (unheated, preferably less than 400, or 300, or 200° C.) feedstock with the higher temperature reactor effluent. This allows for the recovery of a portion of the thermal energy generated by the heater. It also begins the cool-down of the reactor effluent prior to refrigeration in the cooler. This approach is commonly practiced in other chemical processes, but due to the high temperatures in steam cracking this is typically practiced in ethylene production. The electrochemical membrane operates at low temperatures, and the reactions cease once hydrocarbon exits the reactor. This allows for the reuse of thermal energy, lowering the amount of fuel gas consumed by the process.

In any embodiment the heater may be a conventional furnace such as a fired heater, burning fuel gas to raise the temperature of the total charge to reaction temperature.

In any embodiment the cooler is used to cool down the reactor effluent and/or refrigerate the reactor effluent from the membrane reactor in advance of fractionation. The cooler may be a refrigeration unit in which cooling energy is provided by evaporation of a liquid phase refrigerant. Because the membrane reactor runs at higher pressure, the cooling duty is reduced and it may be possible to use a lower cost single stage propylene refrigeration system (versus ethylene propylene used in steam cracking).

In any embodiment the deethanizer column (or “deethanizer”) separates a target alkene, likely with its derivative alkane, from heavier (higher molecular weight) alkanes, such as separating ethane and ethylene (overheads) from C3+ hydrocarbon (bottoms). The C3+ stream consists of any heavier hydrocarbons found in the feed, as well as hydrocarbons which are generated in or downstream of the reactor. If C3 and above hydrocarbon formation is sufficiently low, it is possible that this column could be placed outside of the ethane recycle loop. Heavies (C3 and above) would then be pulled from only the fresh feed to the apparatus.

In any embodiment the C2 splitter column (or “C2 splitter”) separates a target alkene from its derivative alkane, such as separating ethylene product (overheads) from unconverted ethane. The ethylene product is pressurized and sent to sales or directly to a polymerization unit. The recovered ethane can be sent to a recycle pump, increasing its pressure prior to mixing with the fresh feed.

A key difference between the apparatus and process of the present disclosure and conventional steam cracking is the ability to operate the reactor at pressure. A conventional ethane cracking reactor operates at about 40 psig, while downstream reactions and separation may require pressure approaching 400 psig. Achieving the high pressure required for separation a multiple stages of compression, which requires significant equipment investment and energy input. The proton conducting electrochemical reactor disclosed herein avoids these costs, by operating at a higher pressure than is feasible for a conventional steam cracker. By running the reaction at pressure above the first separation step, it may be possible to avoid compressing the feed or other hydrocarbon lines at all.

The product composition from this reactor is distinctly different from steam cracking. In a conventional steam cracking plant, separating hydrogen and methane from heavier hydrocarbons requires high pressure and low temperature. The membrane reactor disclosed herein selectively removes hydrogen and thus requires no additional separation. Methane is not formed at these low temperatures and the elimination of these light gases reduces the severity of the separation units. However, in any embodiment, there may be a need for methane separation as described herein, as an appreciable concentration is expected in the fresh feeds useful for the process described. In any embodiment, such separation is performed prior to any hydrocarbon feed entering the membrane reactor, using a small stripping column that can make use of the inherent high pipeline pressure (e.g., 600 to 1200 psig).

Another benefit of the present process is the recovery of heat from the reactor effluent. Alkenes such as ethylene are highly reactive, and in a conventional steam cracker the hydrocarbon must be rapidly cooled to prevent secondary reactions. This heat is typically removed by circulating/flowing water, generating steam. In any embodiment herein, in the membrane reactor the lower temperature limits the potential for ethylene to react. By recovering effluent heat, fuel consumption which would otherwise be needed to heat the feed to a desirable reaction temperature is reduced.

Alternatively, if conversion of alkanes to alkenes is sufficiently high, it is possible that this recycle stream will cool below the point where energy recovery is feasible.

In any embodiment, a minimum of 30% per pass alkane (e.g., ethane) conversion is achieved for the process disclosed herein. The continuous flow rate of the hydrocarbon feed through the membrane reactor can be controlled to influence this. Preferably, the energy input to this dehydrogenation apparatus could be reduced by 50% relative to a conventional steam cracking unit.

Having elucidated the various features of the inventive process and apparatus, described here in numbered paragraphs is:

P1. A process comprising (or consisting of, or consisting essentially of) continuously contacting an alkane feed at a temperature within a range from 300, or 350, or 400° C. to 450, or 500, or 600° C. and a pressure within the range from 50, or 100, or 200, or 250, or 300 psig to 400, or 450, or 500 psig with one or more reactive ceramic membranes to form an alkene with some remaining alkane and hydrogen, the hydrogen in physical isolation from the alkane and alkene, separating the alkene from the remaining alkane, and recycling the alkane to contact the reactive ceramic membrane.
P2. The process of numbered paragraph 1, wherein the alkene and remaining alkane are cooled to a temperature with a range from −30, or −20, or −10, or 0° C. to 10, or 20, or 30, or 40° C. prior to separating the alkene from the remaining alkane.
P3. The process of numbered paragraphs 1 or 2, wherein the separating step comprises a deethanizer column followed by a C2 splitting column.
P4. The process of any one of the previous numbered paragraphs, wherein the alkane feed is at a pressure within a range from 400, or 500, or 600 psig to 1200, or 1400, or 1800 psig, and a temperature within a range from 0, or 10, or 20° C. to 40, or 50, or 80° C.
P5. The process of any one of the previous numbered paragraphs, wherein the alkane feed comprises at least 80, or 85, or 90, or 95 wt %, by weight of the feed, or ethane, the remaining portion comprising methane, propylene, and butane.
P6. The process of any one of the previous numbered paragraphs, wherein the alkane feed is first passed to a demethanizer column prior to contacting with the reactive ceramic membrane.
P7. The process of any one of the previous numbered paragraphs, wherein the reactive ceramic membrane comprises (or consists essentially of) one or more proton-conducting electrolyte films, one or more porous anode supports and one or more porous cathodes.
P8. The process of numbered paragraph 7, wherein the alkane is fed to the anode(s) and an electrical field is applied across the one or more layers of the reactive ceramic membrane in order to electrochemically deprotonate the alkane to produce the corresponding alkene.
P9. The process of numbered paragraph 7, wherein the reactive ceramic membranes comprise Group 2-Rare Earth complex oxides.
P10. The process of any one of the previous numbered paragraphs, wherein contacting takes place in a membrane reactor comprising the one or more reactive ceramic membranes; wherein the membrane reactor comprises one or more tubes lined with and/or comprising the reactive ceramic membranes, and/or layered throughout the tube(s) therein.
P11. An apparatus comprising (or consisting of, or consisting essentially of) a membrane reactor comprising one or more reactive ceramic membranes, the membrane reactor having at least one inlet for the introduction of heated alkane feed, at least one first outlet for the removal of hydrogen, and at least a second outlet for the recirculation of unreacted alkane and alkene product first to a cooler then to at least one separating column to allow recirculation of alkane back to the membrane reactor.
P12. The apparatus of paragraph 11, also having a demethanizer column upstream of the inlet.
P13. The apparatus of paragraph 12, also having a feed-product exchanger upstream of the inlet and downstream of the demethanizer column.
P14. The apparatus of any one of numbered paragraphs 11 to 13, wherein there are two separating columns downstream of the cooler.
P15. The apparatus of numbered paragraph 14, wherein one of the columns is a deethanizer column, and the other column is a C2 splitter column.

As used herein, the term “consisting essentially of” with respect to a process or apparatus means that the claimed process or apparatus may include some additional minor steps (or means) of routing feed or changing/influencing temperature or pressure, but no change that will influence the major process steps (or components) of heating, reaction (contacting), cooling, and one or more separation steps to obtain the target alkene (preferably ethylene) and hydrogen.

All documents described herein are incorporated by reference for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby.

1. A process comprising continuously contacting an alkane feed at a temperature within a range from 300° C. to 600° C. and a pressure within the range from 50 psig to 500 psig with one or more reactive ceramic membranes to form an alkene with some remaining alkane and hydrogen, the hydrogen in physical isolation from the alkane and alkene, separating the alkene from the remaining alkane, and recycling the alkane to contact the reactive ceramic membrane.

2. The process of claim 1, wherein the alkene and remaining alkane are cooled to a temperature with a range from −30° C. to 40° C. prior to separating the alkene from the remaining alkane.

3. The process of claim 1, wherein the separating step comprises a deethanizer column followed by a C2 splitting column.

4. The process of claim 1, wherein the alkane feed is at a pressure within a range from 400 psig to 1800 psig, and a temperature within a range from 0° C. to 80° C.

5. The process of claim 1, wherein the alkane feed comprises at least 80 wt %, by weight of the feed, or ethane, the remaining portion comprising methane, propylene, and butane.

6. The process of claim 1, wherein the alkane feed is first passed to a demethanizer column prior to contacting with the reactive ceramic membrane.

7. The process of claim 1, wherein the reactive ceramic membrane comprises (or consists essentially of) one or more proton-conducting electrolyte films, one or more porous anode supports and one or more porous cathodes.

8. The process of claim 7, wherein the alkane is fed to the anode(s) and an electrical field is applied across the one or more layers of the reactive ceramic membrane in order to electrochemically deprotonate the alkane to produce the corresponding alkene.

9. The process of claim 7, wherein the reactive ceramic membranes comprise Group 2—Rare Earth complex oxides.

10. The process of claim 1, wherein contacting takes place in a membrane reactor comprising the one or more reactive ceramic membranes; wherein the membrane reactor comprises one or more tubes lined with and/or comprising the reactive ceramic membranes, and/or layered throughout the tube(s) therein.

11. An apparatus comprising a membrane reactor comprising one or more reactive ceramic membranes, the membrane reactor having at least one inlet for the introduction of heated alkane feed, at least one first outlet for the removal of hydrogen, and at least one second outlet for the recirculation of unreacted alkane and alkene product first to a cooler then to at least one separating column to allow recirculation of alkane back to the membrane reactor.

12. The apparatus of claim 11, also having a demethanizer column upstream of the inlet.

13. The apparatus of claim 11, also having a demethanizer column downstream of the inlet.

14. The apparatus of claim 12, also having a feed-product exchanger upstream of the inlet and downstream of the demethanizer column.

15. The apparatus of claim 11, wherein there are two separating columns downstream of the cooler.

16. The apparatus of claim 15, wherein one of the columns is a deethanizer column, and the other column is a C2 splitter column.
